(19) United States
(12) Patent Application Publication
(10) Pub. No.: US 2007/0048743 A1
(43) Pub. Date: Mar. 1, 2007

(54) Methods and compositions for assessing candidate aCGH probe nucleic acids

(76) Inventors: Nicholas M. Sampas, Loveland, CO (US); Michael T. Barrett, Loveland, CO (US)

Correspondence Address: AGILENT TECHNOLOGIES INC., INTELLECTUAL PROPERTY ADMINISTRATION, M/S DU404, P.O. BOX 7599, LOVELAND, CO, 80537-0599, US

(21) Appl. No.: 11/213,561

(22) Filed: Aug. 26, 2005

(51) Int. Cl. C40B 30/06; C40B 40/08; C12Q 1/68

(52) U.S. Cl. 435/6

(57) ABSTRACT

Methods for evaluating surface-bound polynucleotides, e.g., candidate aCGH probe nucleic acids, are provided. Specifically, the methods involve contacting an array of surface-bound polynucleotides with a validation nucleic acid composition and assessing binding of the surface-bound polynucleotides. The methods may be used to screen for surface bound polynucleotides that have desirable binding characteristics, e.g., suitability for use in array-based comparative genomic hybridization assays.

### INTRODUCTION

### Background of the Invention

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, develop predictors of disease outcomes, improve prognosis of therapeutic response, and permit earlier tumor detection. In addition, perinatal genetic problems frequently result from loss or gain of chromosome segments such as trisomy 21 or the micro deletion syndromes. Thus, methods of prenatal detection of such abnormalities can be helpful in early diagnosis of disease.

Comparative genomic hybridization (CGH) is one approach that has been employed to detect the presence and identify the location of amplified or deleted sequences. In one implementation of CGH, genomic DNA is isolated from normal reference cells, as well as from test cells (e.g., tumor cells). The two nucleic acids are differentially labeled and then simultaneously hybridized in situ to metaphase chromosomes of a reference cell. Chromosomal regions in the test cells which are at increased or decreased copy number relative to the reference cells can be identified by detecting regions where the ratio of the signals from the two distinguishably labeled nucleic acids is altered. For example, those regions that have been decreased in copy number in the test cells will show relatively lower signal from the test nucleic acids than the reference compared to other regions of the genome. Regions that have been increased in copy number in the test cells will show relatively higher signal from the test nucleic acid.

In a recent variation of the above traditional CGH approach, the immobilized chromosome elements have been replaced with a collection of solid support surface-bound polynucleotides, e.g., an array of BAC (bacterial artificial chromosome) clones or cDNAs. Such approaches offer benefits over immobilized chromosome approaches, including a higher resolution, as defined by the ability of the assay to localize chromosomal alterations to specific areas of the genome.

Despite great interest in CGH technology, methods for empirically evaluating and identifying suitable surface-bound polynucleotides for use in this technology are limited.

Accordingly, a great need exists for methods for evaluating surface-bound CGH probe nucleic acids.

### RELEVANT LITERATURE

U.S. patents of interest include: U.S. Pat. Nos. 6,465,182; 6,335,167; 6,251,601; 6,210,878; 6,197,501; 6,159,685; 5,965,362; 5,830,645; 5,665,549; 5,447,841 and 5,348,855. Also of interest are published United States Application Serial Nos. 20020006622; 20040241658 and 20040191813, as well as published PCT application WO 99/23256. Articles of interest include: Pollack et al., Proc. Natl. Acad. Sci. (2002) 99: 12963-12968; Wilhelm et al., Cancer Res. (2002) 62: 957-960; Pinkel et al., Nat. Genet. (1998) 20: 207-211; Cai et al., Nat. Biotech. (2002) 20: 393-396; Snijders et al., Nat. Genet. (2001) 29:263-264; Hodgson et al., Nat. Genet. (2001) 29:459-464; and Trask, Nat. Rev. Genet. (2002) 3: 769-778

### SUMMARY OF THE INVENTION

Methods for evaluating surface-bound polynucleotides, e.g., candidate aCGH probe nucleic acids, are provided. Specifically, the methods involve contacting an array of surface-bound polynucleotides with a validation nucleic acid composition and assessing binding of the surface-bound polynucleotides. In certain embodiments, the validation nucleic acid composition is a portion of an initial clone library that spans at least a portion of a genome; while in other embodiments the validation nucleic acid composition is a portion of a genome in which all constituent members have substantially the same value for at least one physical parameter. The methods may be used to screen for surface bound polynucleotides that have desirable binding characteristics, e.g., suitability for use in array-based comparative genomic hybridization assays. Kits and computer programming for use in practicing the subject methods are also provided.

### BRIEF DESCRIPTION OF THE FIGURES

FIG. 1 is a schematic representation of an embodiment of the subject methods.

FIG. 2 is a flow chart of a process for validating candidate probes by target hybridization experiments for selecting probes for CGH arrays in accordance with the invention.

FIG. 3 is a flow chart of a process of probe selection utilizing probe selection parameters for CGH in accordance with an embodiment invention.

### DEFINITIONS

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length, or longer, e.g., up to 500 nt in length or longer. However, in representative embodiments, oligonucleotides are synthetic and, in certain embodiments, are under 50 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of oligonucleotide target elements employed herein are present on a surface of the same planar support, e.g., in the form of an array.

A “surface-bound polynucleotide with desirable binding characteristics”, as discussed in greater detail below, refers to a surface-bound polynucleotide that has properties that make it suitable for array-based comparative genome hybridization experiments. Such polynucleotides usually exhibit an observed binding behavior that is similar to an expected binding behavior. For example, if binding of a surface-bound polynucleotide to its target sequence is expected to be linear then that polynucleotide is a surface-bound polynucleotide with desirable binding characteristics if it actually exhibits linear binding.

The phrase “labeled population of nucleic acids” refers to mixture of nucleic acids that are detectably labeled, e.g., fluorescently labeled, such that the presence of the nucleic acids can be detected by assessing the presence of the label. A labeled population of nucleic acids is “made from” a chromosome composition, the chromosome composition is usually employed as template for making the population of nucleic acids.

A “non-cellular chromosome composition”, as will be discussed in greater detail below, is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell. The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm, a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic riethods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” will be referenced as a moiety in a mobile phase (typically fluid), to be detected by “surface-bound polynucleotides” which are bound to the substrate at the various regions. These phrases are synonymous with the terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as signals (e.g., electrical, optical, radio signals, etc.) over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M CaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M CaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M CaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “pre-determined” refers to an element whose identity or composition is known prior to its use. For example, a “pre-determined chromosome composition” is a composition containing chromosomes of known identity. An element may be known by name, sequence, molecular weight, its function, or any other attribute or identifier.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The term “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional application, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

“Contacting” means to bring or put together. As such, a first item is contacted with a second item when the two items are brought or put together, e.g., by touching them to each other.

A “probe” means a polynucleotide which can specifically hybridize to a target nucleotide, either in solution or as a surface-bound polynucleotide.

The term “validated probe” means a probe that has been passed by at least one screening or filtering process in which experimental data related to the performance of the probes was used as part of the selection criteria.

“In silico” means those parameters that can be determined without the need to perform any experiments, by using information either calculated de novo or available from public or private databases.

The term “genome” refers to all nucleic acid sequences (coding and non-coding) and elements present in or originating from any virus, single cell (prokaryote and eukaryote) or each cell type and their organelles (e.g. mitochondria) in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

By “genomic source” is meant the initial nucleic acids that are used as the original nucleic acid source from which the solution phase nucleic acids are produced, e.g., as a template in the labeled solution phase nucleic acid generation protocols described in greater detail below.

The genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in certain embodiments, the genomic source may comprise a portion of the genome, e.g., one or more specific chromosomes or regions thereof, such as PCR amplified regions produced with a pairs of specific primers.

A given initial genomic source may be prepared from a subject, for example a plant or an animal, which subject is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that they may be about 1 Mb or smaller, e.g., less than about 500 Kb, etc.

In certain embodiments, the genomic source is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse genomic sources. In certain embodiments, a set of nucleic acid sequences within the genomic source is complex, as the genome contains at least about 1×10⁸ base pairs, including at least about 1×10⁹ base pairs, e.g., about 3×10⁹ base pairs.

Where desired, the initial genomic source may be fragmented in the generation protocol, as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

Where desired, the initial genomic source may be amplified as part of the solution phase nucleic acid generation protocol, where the amplification may or may not occur prior to any fragmentation step. In those embodiments where the produced collection of nucleic acids has substantially the same complexity as the initial genomic source from which it is prepared, the amplification step employed is one that does not reduce the complexity, e.g., one that employs a set of random primers, as described below. For example, the initial genomic source may first be amplified in a manner that results in an amplified version of virtually the whole genome, if not the whole genome, before labeling, where the fragmentation, if employed, may be performed pre- or post-amplification.

### Description of the Specific Embodiments

Methods for evaluating surface-bound polynucleotides, e.g., candidate aCGH probe nucleic acids, are provided. Specifically, the methods involve contacting an array of surface-bound polynucleotides with a validation nucleic acid composition and assessing binding of the surface-bound polynucleotides. In certain embodiments, the validation nucleic acid composition is a portion of an nitial clone library that spans at least a portion of a genome; while in other mbodiments the validation nucleic acid composition is a portion of a genome in which all constituent members have substantially the same value for at least one physical parameter. The methods may be used to screen for surface bound polynucleotides that have desirable binding characteristics, e.g., suitability for use in array-based comparative genomic hybridization assays. Kits and computer programming for use in practicing the subject methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

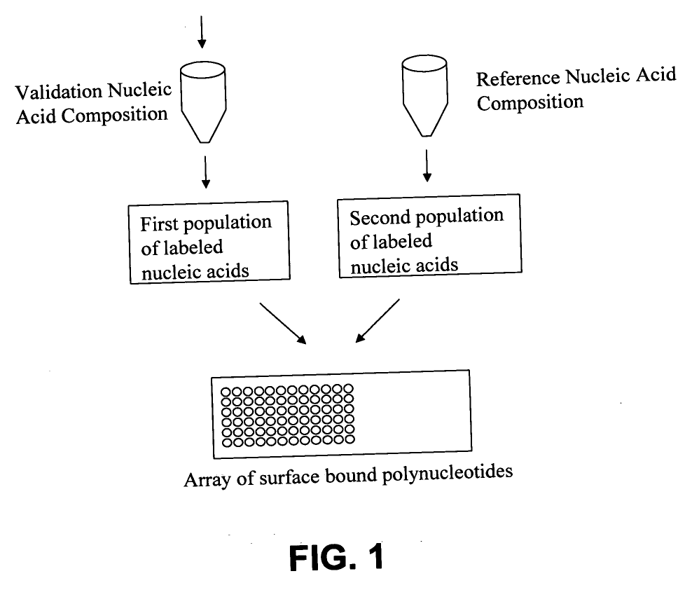

As summarized above, the present invention provides methods for evaluating surface-bound polynucleotides. In many representative embodiments, the subject methods include contacting an array of surface-bound polynucleotides with a labeled population of nucleic acid acids produced from a validation nucleic acid composition. Following contact, signals obtained from the surface-bound polynucleotides are compared with a reference, e.g., a previously or concurrently determined control set of values, to evaluate the binding characteristics of one or more of the surface-bound polynucleotides. With reference to FIG. 1, showing an exemplary embodiment of the invention, the methods may involve obtaining a validation nucleic acid composition and a reference nucleic acid composition, making first and second populations of labeled nucleic acids using from these initial compositions, and contacting the first and second labeled populations of nucleic acids with an array of surface bound polynucleotides, e.g., that includes one or more candidate aCGH probe nucleic acids. The resultant signals are obtained and compared, and an evaluation of the surface made polynucleotides is made based on this comparison.

In further describing the present invention, validation and reference nucleic acid compositions, as well as arrays of surface-bound polynucleotides are described first, followed by a more in-depth description of the subject methods. Finally, representative kits and computer programming for use in practicing thesubject methods will be discussed.

Validation Nucleic Acid Compositions

As mentioned above, the invention employs validation nucleic acid compositions. A validation nucleic acid composition is a composition made up of “target” nucleic acids, where the composition represents a portion or fraction of a genome of interest, such that it does not include all of the sequence information in the genome of interest. In certain embodiments, a validation nucleic acid composition includes no more than about 50%, such as no more than about 40%, including no more than about 33%, no more than about 25% or less of the sequence information or content of the genome of interest. As such, a validation nucleic acid composition is one that has a reduced complexity as compared to its corresponding genome of interest. A given validation composition is considered to be of reduced complexity as compared to its corresponding genome if its complexity is less than about 50%, such as lass than about 40%, including less than about 33%, less than about 25% of the complexity of the genome of interest, e.g., as determined using the nucleic acid composition complexity test detailed in Published United States Patent Application 20040191813. As such, a validation nucleic acid composition is a population of nucleic acids that may be viewed as a subset of a genome of interest.

A given validation composition is deterministically known or characterizable, such that the sequence content of the target nucleic acids that make up the composition can be readily established. In other words, the sequences of target nucleic acids, as well as copy numbers thereof, that make up a given validation nucleic acid composition are known, or readily determinable, e.g., based on the protocol employed to make the validation composition. The sequence information/copy number of the target nucleic acids that make up a given validation composition is, in many embodiments, collectively known for that composition. By “collectively known” is meant that, in view of the way in which a given validation composition is prepared, it is known or readily determinable that a composition includes target nucleic acids having specific sequences, as well as how much of a given specific target nucleic acid is present in the composition.

In a given validation nucleic acid composition, the total number of different or distinct nucleic acids may vary. Generally, the subject compositions include at least about 100, as at least about 10,000, including at least about 100,000 distinct target nucleic acids, where any two given target nucleic acids are considered distinct if they comprise a stretch of at least 20 nt in which the sequence similarity does not exceed 98%, as determined by FASTA (default settings). The length of the target nucleic acids that make up a given validation composition may vary, but in representative embodiments ranges from about 1 Kb to about 250 Mb, such as from about 20 K to about 500 Kb, exclusive of any vector material in which the target nucleic acid may be present. The total amount of nucleic acid in a given composition may vary, but in representative embodiments ranges from about 1% to about 90%, such as from about 10% to about 50% (by weight).

As indicated above, two representative validation nucleic acid compositions that find use in the subject methods are: (i) a validation composition that is a portion of an initial clone library, where the clone library spans at least a portion of a genome; and (ii) a portion of a genome in which all constituent members have substantially the same value for at least one physical parameter. Each of these representative validation compositions is now reviewed further in greater detail.

In certain representative embodiments, the validation nucleic acid composition is made up of a portion of an initial clone library that spans at least a portion of a genome. The term “clone library” is used to refer to a collection of multiple distinct clones of regions (or compilations thereof) of a genome of interest. As such, the clone libraries of the subject invention may be viewed as collections of cloned genomic DNA. In certain embodiments, the clones may be contigs (groups of clones representing overlapping regions of the genome of interest). As such, in certain embodiments the initial clone library is a contig library.

In representative embodiments, the initial clone library comprises clones that represent approximately uniformly spaced regions of the genome. In certain embodiments, the uniformly spaced regions are overlapping, while in other embodiments the uniformly spaced regions are non-overlapping, such that the initial clone library does not include a clone for every region or portion of the genome of interest. In certain embodiments, the constituent members of the clone library are nucleic acids representative of locations distributed over the entire genome, such that the library is considered to span the entire genome. In yet other embodiments, the clones collectively include nucleic acids that correspond to locations distributed over only a portion of the genome, e.g., a subset of all of the chromosomes, a single chromosome, a portion of a chromosome, etc., such that the library spans only a portion of the genome.

Where the initial clone library is made up of non-overlapping clones of regions of the genome, the average spacing may vary, where in certain embodiments, the average spacingis at least about 500 kb, such as at least about 250 kb, at least about 200 kb, at least about 150 kb, including at least about 100 kb, and sometimes at least about 50 kb, at least about 25 kb, at least about 10 kb or higher. Of interest in certain embodiments are resolutions ranging from about 20 kb to about 100 kb, such as 30 kb to about 100 kb, including from about 40 kb to about 75 kb. By average spacing is meant the spacing on the genome between a common reference point, e.g., start positions of the sequences found in the initial clone library.

As indicated above, in some embodiments (e.g., in libraries that span the genome without any intervening regions) all sequences within the genome can be present on the array. In certain embodiments, the resolution of the array is with respect to at least a portion of the genome, and may be about every 1 kb, about every 2 kb, about every 5 kb, about every 10 kb, as well as the numbers provided above. The resolution of the oligonucleotides on the array may differ from region to region. The spacing between different locations of the genome that are represented in the library may also vary, and may be uniform, such that the spacing is substantially the same, if not the same, between sampled regions, or non-uniform, as desired.

In representative embodiments, the initial clone library is made up of clones of genomic DNA present in a vector, where the vector may be common to all the constituent members of the library. The choice of vector may vary and may be chosen, at least in part, in view of the size of the clone to be accommodated by the vector. Representative vectors of interest include, but are not limited to: plasmid vectors, viral vectors and artificial chromosome vectors, including but not limited to: bacterial artificial chromosomes (BACs), yeast artificial chromosomes (YACs), P1 artificial chromosomes (PACs), etc.

The initial clone library that may be employed in these representative embodiments may be obtained using any convenient protocol. In certain embodiments, the initial clone library may be prepared de novo, e.g., using an initial genome or genomic source and conventional library generation protocols. Alternatively, the initial clone library may be obtained from a source or vendor thereof. A number of different libraries suitable for use as initial clone libraries in the subject methods have been reported in the art, including but not limited to: the BAC library reported in lshkanian et al., Nature Genetics (February 2004) 10:1038; Cowell et al., British Journal of Cancer (2004), Shaw-Smith et al., Journal of Med Genetics (2004) and the like.

As indicated above, the validation nucleic acid composition of these embodiments is made up of a selection or portion of the constituent members of the initial library. In other words, the validation nucleic acid composition is a subpopulation of the initial clone library. In representative embodiments, the number percent of constituent members of the initial clone library present in the validation nucleic acid composition is less than about 90%, such as less than about 50%, including less than about 10%. In representative embodiments, the validation nucleic acid composition is a composition that is the product of a rational selection of the members of the initial clone library, where criteria employed in selection of the members may include: predicted presence or absence of sequence complementary to a given candidate probe nucleic acid of interest, predicted presence or absence of a nucleic acid that may participate in undesirable cross-hybridization, predicted presence or absence of probes that have undesirable hybridization kinetic properties, and the like. This selection may be performed, at least in part, using in silico protocols, as desired.

In certain of these embodiments, the validation nucleic acid composition is a collection of target nucleic acids that is produced by first separating or dividing an initial clone library into two or more subpopulations, e.g., at least one subpopulation of chosen or selected members and at least one subpopulation of non-selected members; and then selecting the subpopulation predicted to include a target that corresponds to a candidate aCGH probe nucleic acid of interest as the validation nucleic acid composition. By “corresponds” is meant that the target, or a labeled derivative thereof, has a sequence that, at least in silico, predicts a binding interaction between the two under hybridization conditions, e.g., stringent hybridization conditions. In many embodiments, corresponds means that the sequence of the target, or the complementary sequence thereof, is substantially the same as, if not identical to, a sequence found in the probe molecule. Substantially the same means that for a length of at least about 10 nt, sequence identity (e.g., as determined using BLAST and default settings) is at least about 80%, if not 90%, 95%, 99%, or higher.

As indicated above, in certain representative embodiments, the validation nucleic acid composition is made up of a portion of a genome in which all constituent members of the composition have substantially the same value for at least one physical parameter. By physical parameter is meant a non-sequence parameter, i.e., a parameter that does not include specific sequence information for a given target nucleic acid. Representative physical parameters of interest include, but are not limited to: length, mass and charge-to-mass ratio. In particular embodiments of interest, the physical parameter is length.

As indicated above, with respect to the common physical parameter that characterizes a given validation nucleic acid composition of these embodiments, the value of the physical parameter will be substantially the same among all members of the validation composition, such that the magnitude of any difference in value between any two members of the composition does not exceed about 10-fold. For example, where the physical parameter is length, magnitude of any difference in the lengths of any two constituent members of the validation nucleic acid composition does not exceed about 200 Mb, and in certain representative embodiments does not exceed about 1 Mb, e.g., does not exceed about 100 kb, including does not exceed about 100 nt.

The validation composition of these embodiments may be produced using any convenient protocol. For example, the validation composition may be produced from an initial genomic source, e.g., as described above, by subjecting the initial genomic source to a fragmenting or cleavage protocol. As such, the initial genomic source may be fragmented, as desired, to produce a fragmented genomic source, where the molecules have a desired average size range, e.g., up to about 10 Kb, such as up to about 1 Kb, e.g., where particular size ranges of interest include: from about 0.1 kb to about 50 kb_, such as from about 1 kb to about 10 kb. Fragmentation is generally achieved in a manner such that the sequences of the resultant fragments are predictable, at least to some extent. As such, fragmentation is generally performed in a manner that cuts or cleaves the initial genomic source at known locations.

In representative embodiments, an enzymatic cleavage protocol is employed, in which the initial genomic source is contacted with one or more nucleases, e.g., restriction endonucleases, which cleave the nucleic acids of the initial genomic source into fragments of desired size. In certain embodiments, a single frequently cutting enzyme may be employed, such as CVIJI or DNAse. In certain embodiments, a combination of two or more restriction endonulceases are employed, where the two or more restriction endonucleases that are employed are selected or chosen to cleave the initial dsDNA molecule into fragments of a predetermined size. In such embodiments, the number of restriction endonucleases that are employed may vary, e.g., from about 2 to about 10, such as from about 3 to about 8, including from about 3 to about 7, e.g., 3, 4, 5 or 6. In these embodiments, the plurality of restriction endonucleases are chosen based on the predicted frequency of their respective recognition sites in the initial genomic source to be cleaved, so that the combined action of the plurality of nucleases at least theoretically results in fragments of a desired predetermined size and/or sequence content. As such, a collection or plurality of endonucleases may be chosen that at least theoretically will cleave the genomic source into fragments that have a predicted predetermined size ranging from about 0.1 kb to about 10 kb. The collection or plurality of endonucleases that is employed may vary greatly, where suitable collections or combinations of enzymes can readily be determined by those of skill in the art based on known recognition sites, predicted frequency in the dsDNA to be cleaved, etc. Specific enzymes of interest include, but are not limited to: NotI, MluI, HindIII, BamHI, EcoRI, AluI, RsaI, DpnI, etc., and specific enzyme combinations of interest include, but are not limited to: AluI/RsaI, BamHI/EcoRI, NotI/HindIII, and the like.

In these embodiments, following production of the population of fragment nucleic acids from the initial genomic source, the population of fragmented nucleic acids is separated into two or more subpopulations based on at least one physical parameter, e.g., length, in a manner such that each member of a given subpopulation has substantially the same value with respect to the physical parameter. The particular manner of separation may vary, and will be chosen at least partially with respect to the physical parameter of interest. Representative separation protocols that may be employed include, but are not limited to: electrophoretic separation protocols (e.g., gel electrophoresis, pulse-field electrophoresis, capillary electrophoresis, etc.); chromatographic separation protocols (e.g., liquid chromatography, gas chromatography, etc.); mass-spectroscopy; and the like.

In representative embodiments where the physical parameter of interest is length, electrophoretic protocols are conveniently employed. Conveniently, a fragment population of an initial genomic source is “run” on a gel to produce a number of different bands of different molecular weight range in the gel, where the resultant bands are representative of length. The resultant bands may then be excised from the gel, and the nucleic acids in the bands separated from the gel media to produce a plurality of size separated fractions of the fragmented composition run on the gel. Protocols for performing each of these steps are standard in the art. In this manner, the fragmented population is separated into two or more subpopulations, where each subpopulation is made up of target nucleic acids that have substantially the same length.

Because the fragmentation protocol employed to fragment the initial genomic source cut the initial genomic source at known locations such that the sequence content of all of the resultant fragments is collectively known, the sequence content of each of the subpopulations is also collectively known. As such, the predicted sequences of each of the targets in a given subpopulation are known or readily determined.

Following production of the two or more different subpopulations from the fragmented product of the initial genomic source, the subpopulation predicted to include a sequence that will bind to the candidate aCGH probe of interest is selected as the validation nucleic acid composition.

Reference Nucleic Acid Compositions

As mentioned briefly above and as shown in FIG. 1, a validation nucleic acid composition may be employed in conjunction with a reference nucleic acid composition, e.g., where a reference is obtained concurrently with data from the validation composition. As will be described in greater detail below, in certain representative embodiments the results obtained using a particular validation nucleic acid composition are compared to the results obtained using a reference nucleic acid composition. In some embodiments, the validation nucleic acid composition and the reference nucleic acid composition have common genomic sequences present in equal concentrations. These sequences can consist of a portion of a chromosome, an entire chromosome, or multiple chromosomes. These enable the direct sample comparisons by providing signal intensity calibration across the two samples.

In certain embodiments, reference compositions may be made directly from a cell, by isolating a chromosomal extract from the cell. If it is desirable, a reference composition having a composition that is identical to that of a particular cell may be “reconstituted” using isolated chromosomes. In certain embodiments, the reference nucleic acid composition is a genomic source from a normal cell, in which the genomic content of the cell is characterized.

In general, the reference composition may contain genomic material from any cell of an organism with a genome e.g., yeast, plants and animals, such as fish, birds, reptiles, amphibians and mammals. In certain embodiments, reference compositions containing genomic material from mice, rabbits, primates, or humans, etc, can be made and used. Suitable cells that may be used as a source of genomic material for use as reference compositions include: monkey kidney cells (COS cells), human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, TCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); NIH/3T3 cells (ATCC CRL-1658); and mouse L cells (ATCC CCL-1). Additional cells (e.g. human lymphocytes) and cell lines will become apparent to those of ordinary skill in the art, and a wide variety of cell lines are available from the American Type Culture Collection, 10801 University Boulevard, Manassas, Va. 20110-2209.

Array Platforms

Array platforms for performing the subject methods are generally well known in the art (e.g., see Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described herein in any great detail. In general, arrays suitable for use in performing the subject methods contain a plurality (i.e., at least about 100, at least about 500, at least about 1000, at least about 2000, at least about 5000, at least about 10,000, at least about 20,000, usually up to about 100,000 or more) of addressable features that are linked to a usually planar solid support. Features on a subject array usually contain a polynucleotide that hybridizes with, i.e., binds to, genomic sequences from a cell. Accordingly, such “comparative genome hybridization arrays”, for short “CGH arrays” typically have a plurality of different BACs, cDNAs, oligonucleotides, or inserts from phage or plasmids, etc., that are addressably arrayed. As such, CGH arrays usually contain surface bound polynucleotides that are about 10-200 bases in length, about 201-5000 bases in length, about 5001-50,000 bases in length, or about 50,001-200,000 bases in length, depending on the platform used.

In particular embodiments, CGH arrays containing surface-bound oligonucleotides, i.e., oligonucleotides of 10 to 100 nucleotides and up to 200 nucleotides in length, find particular use in the subject methods.

Array platforms of interest include, but are not limited to: U.S. patents of interest include: U.S. Pat. Nos. 6,465,182; 6,335,167; 6,251,601; 6,210,878; 6,197,501; 6,159,685; 5,965,362; 5,830,645; 5,665,549; 5,447,841 and 5,348,855. Also of interest are published United States Application Serial Nos. 20020006622; 20040241658 and 20040191813, as well as published PCT application WO 99/23256.

Methods

The validation nucleic acid compositions described above are generally useful in methods of assessing a surface bound polynucleotide of interest. In general, the methods involve contacting a population of labeled nucleic acids made from a validation nucleic acid composition with an array of surface-bound polynucleotides, and evaluating a surface bound polynucleotide of interest, e.g., a candidate aCGH probe nucleic acid, for binding to the labeled nucleic acids. In certain embodiments, evaluating includes comparing signals to signals obtained from binding a reference composition to the array, e.g., by evaluating binding relative to binding of the polynucleotide of interest to a population of nucleic acids made from a reference nucleic acid composition.

In representative embodiments, the first step is labeling a validation and a reference composition to make two labeled populations of nucleic acids which may be distinguishably labeled, contacting the labeled populations of nucleic acids with at least one array of surface bound polynucleotides under specific hybridization conditions, and analyzing any data obtained from hybridization of the nucleic acids to the surface bound polynucleotides. Such methods are generally well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described herein in any great detail.

In certain embodiments, the validation and reference compositions (or amplification products thereof), are distinguishably labeled using methods that are well known in the art (e.g., primer extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The compositions may be labeled using “distinguishable” labels in that the labels that can be independently detected and measured, even when the labels are mixed. In other words, the mounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Bostan Mass.) and POPRO3 TOPRO3 (Molecular Probes, Eugene, Oreg.). Further suitable distinguishable detectable labels may be found in Kricka et al. (Ann Clin Biochem. 39:114-29, 2002).

The labeling reactions produce a first and second population of labeled nucleic acids that correspond to the validation and reference nucleic acid compositions, respectively. After nucleic acid purification and any pre-hybridization steps to suppress repetitive sequences (e.g., hybridization with Cot-1 DNA), the populations of labeled nucleic acids are contacted to an array of surface bound polynucleotides, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

The labeled nucleic acids can be contacted to the surface bound polynucleotides serially, or, in other embodiments, simultaneously (i.e., the labeled nucleic acids are mixed prior to their contacting with the surface-bound polynucleotides). Depending on how the nucleic acid populations are labeled (e.g., if they are distinguishably or indistinguishably labeled), the populations may be contacted with the same array or different arrays. Where the populations are contacted with different arrays, the different arrays are substantially, if not completely, identical to each other in terms of target feature content and organization.

Standard hybridization techniques (using high stringency hybridization onditions) are used to probe a target nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos.: 6,335,167; 6,197,501; 5,830,645; and 5,665,549; the disclosures of which are herein incorporate by reference.

Generally, comparative genome hybridization methods comprise the following major steps: (1) immobilization of polynucleotides on a solid support; (2) pre-hybridization treatment to increase accessibility of support-bound polynucleotides and to reduce nonspecific binding; (3) hybridization of a mixture of labeled nucleic acids to the surface-bound nucleic acids, typically under high stringency conditions; (4) post-hybridization washes to remove nucleic acid fragments not bound to the solid support polynucleotides; and (5) detection of the hybridized labeled nucleic acids. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between the surface-bound polynucleotides and complementary labeled nucleic acids in a sample. Representative high stringency assay conditions that may be employed in these embodiments are provided above.

The above hybridization step may include agitation of the immobilized polynucleotides and the sample of labeled nucleic acids, where the agitation may be accomplished using any convenient protocol, e.g., shaking, rotating, spinning, and the like.

Following hybridization, the array-surface bound polynucleotides are typically washed to remove unbound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications Ser. No. 09/846125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Accordingly, a pair of validation and reference compositions is labeled to make two populations of labeled nucleic acids, the nucleic acids contacted with an array of surface-bound polynucleotides, and the level of labeled nucleic acids bound to each surface-bound polynucleotide is assessed.

In certain embodiments, a surface-bound polynucleotide is assessed by determining the level of binding of the population of labeled nucleic acids to that polynucleotide. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of labeled nucleic acid to a surface-bound polynucleotide is proportional to the level of bound label, the level of binding of labeled nucleic acid is usually determined by assessing the amount of label associated with the surface-bound polynucleotide.

In certain embodiments, a surface-bound polynucleotide may be assessed by evaluating its binding to two populations of nucleic acids that are distinguishably labeled. In these embodiments, for a single surface-bound polynucleotide of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

By “normalization” is meant that data corresponding to the two populations of nucleic acids are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol. 48(1-2):119-31, 2002, and Hegde et al, Biotechniques. 29:548-554, 2000). Specific examples of normalization suitable for use in the subject methods include linear normalization methods, non-linear normalization methods, e.g., using lowest local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using the data obtained from a support-bound polynucleotide for a chromosome of known concentration in any of the chromosome compositions.

Accordingly, binding of a surface-bound polynucleotide to a labeled population of nucleic acids may be assessed. In most embodiments, the assessment provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof.

In other words, any binding may be expressed as the level of binding of a surface-bound polynucleotide to a labeled population of nucleic acids made from a non-cellular chromosome composition, divided by its level of binding to a labeled population of nucleic acids made from a reference chromosome composition (or vice versa).

Methods of Screening

The methods of assessing described above find use in methods of screening for surface-bound polynucleotides with binding characteristics that make them suitable for use in array-based comparative genome hybridization methods. Accordingly, the invention provides a method of screening in which binding of a candidate surface-bound polynucleotide is assessed using the methods described above, and surface-bound polynucleotides with desirable binding characteristics are identified.

In many embodiments, a surface-bound polynucleotide has desirable binding characteristics if data obtained using that polynucleotide corresponds to data expected for that polynucleotide. For example, candidate surface-bound polynucleotide binding may be assessed in a series of hybridization experiments using populations of labeled nucleic acids made from different validation nucleic acid compositions, as discussed above, and surface-bound polynucleotides may be screened on the basis of their level of binding to the labeled nucleic acids.

In certain embodiments the methods of the invention when candidate probes are screened according to at least one experimentally measurable parameter or property, the experimentally measurable property or parameter is selected from the group consisting of signal intensity, reproducibility of signal intensity, dye bias, susceptibility to non-specific binding, wash stability and persistence of probe hybridization. In embodiments where experimentally validating candidate probe performance is used for probe selection, validating the candidate probes comprises hybridizing the candidate probes to a plurality of target sets, evaluating the candidate probes for a probe property for each target set, and comparing the values for probe property of each candidate probe across a plurality of target sets.

By providing a method of assessing surface-bound polynucleotides, candidate surface-bound polynucleotides may be screened to identify surface-bound polynucleotides with desirable binding characteristics.

### Representative Embodiments

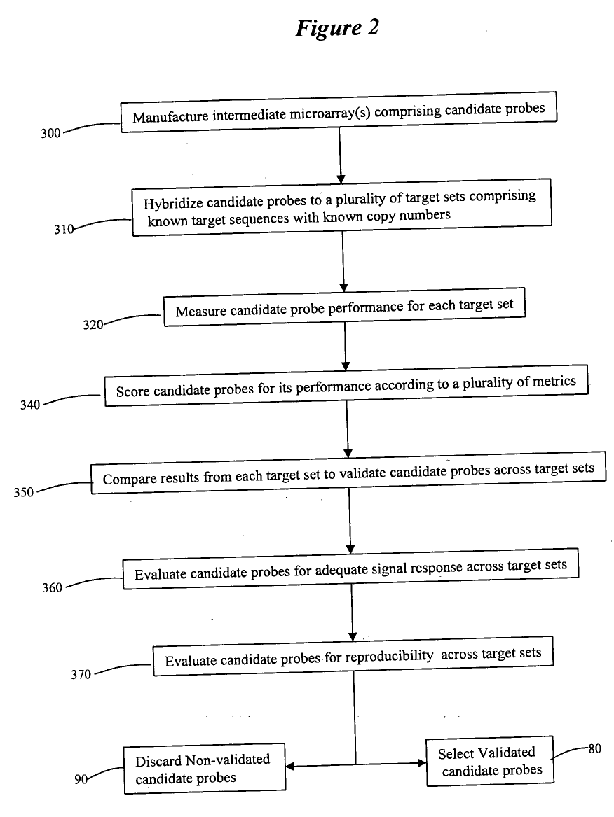

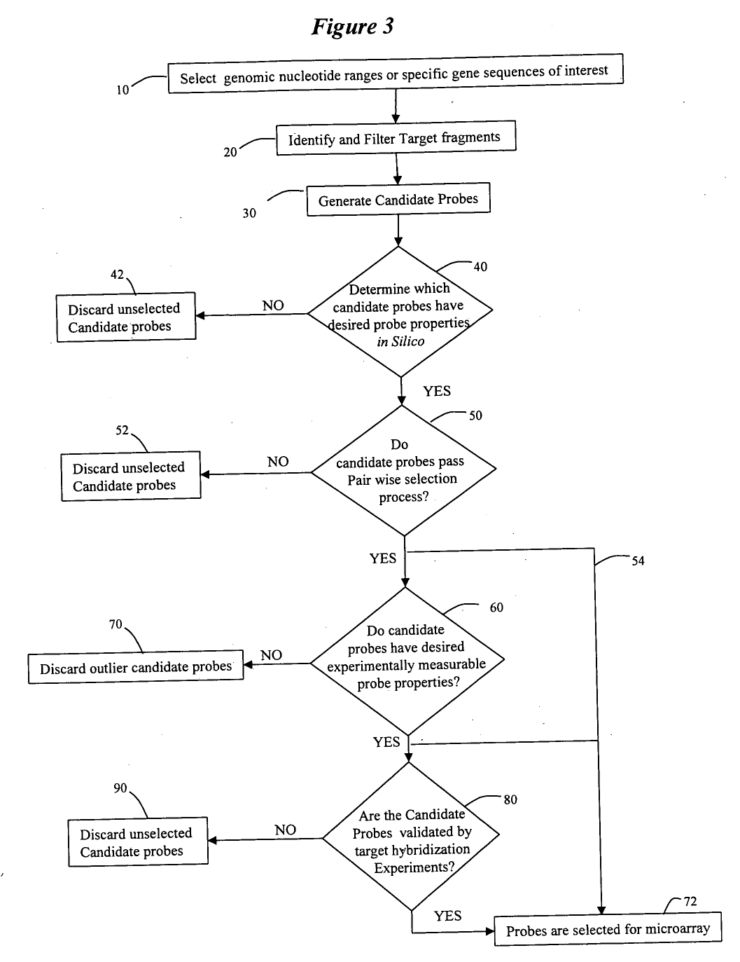

In further illustrating the subject methods, a representative embodiment of using the validation compositions is described in terms of FIG. 2, and then a representative embodiment in which the subject methods are incorporated into an overall probe design protocol that includes in silico steps is reviewed in terms of FIG. 3.

Referring now to FIG. 2, there is shown a flow chart of events useful in determining probe properties of candidate probes directly by experimentation. Direct measurement of probe performance, is determined by validation experiments using samples whose relative copy numbers of complementary genomic sequences are known a priori, e.g., validation and reference compositions as described above. At event 300, candidate probes, which have desirable probe properties determined in silico and/or empirically, are laid out on a prototype array. In certain embodiments, it may be useful to proceed with direct experimentation with limited or no in silico or empirical prior data. The candidate probes are placed on the array using array techniques known to those skilled in the art of microarrays. Array layout protocols include randomization, periodic grid tiling, text-ordered tiling, and serpentine tiling. By making prototype “intermediate” arrays with more probes for a given region of the genomic range of interest, than would be placed on a final array design, those probes that behave best according to some set of metrics for probe performance for that particular region can be selected.

At event 310, the candidate probes on the array are hybridized to various target sets comprising known target sequence with known copy number, e.g., a validation or reference composition, as described above. A target set comprises a quantity of target molecules within the mixture that is deterministically altered, or known to differ in a well-defined way from that of a “normal” target set sample. A plurality of arrays are utilized to test various probe properties for the plurality of target sets.

In general, all subsets of the target sequences are altered in copy number (or deleted altogether) without dramatically altering the composition of the rest of the genome. For example, two normal tissue samples, one from a male tissue or cell line, and another from a female can be analyzed. Both will have the same number of target sequences for each region of every chromosome (notwithstanding the usual polymorphic variations) except the X-chromosome and the Y-chromosome. The male sample has a single copy of the X and Y chromosomes, whereas the female will have two nominally identical copies of the X-chromosome. So the male sample will have ½ the number of copies of the X-chromosome as does the female sample, and the female sample will have no copies of the Y-chromosome targets. Probes for targets on the X-chromosome should display, after normalization, twice as much signal for the female sample as for the male sample. The fractional increase in the log base 2 of the observed signal for a probe, when the copy number of its intended target is doubled, is the “slope” of that probe. Ideally, probes should have a slope of 1.0. Probes with significantly and systematically different slopes are inferior performers, and are issued low “differential response” scores. The same approach can be used with cell lines of known chromosomal copy number variations, where they can be found. It is unlikely that cell lines with alterations spanning the whole human genome can be derived from naturally occurring variations (e.g. diseases). With such a set of samples, multiple measurements analogous to those of male to female signal ratios for each probe can be obtained.

Also, when the known copy number of a particular target sequence in a sample is zero (as, for example, Y-chromosome probes in female samples), any signal observed for that probe must result from cross-hybridization. Probes that show significant signal for samples in which their known copy number is zero are scored low on the “cross-hybridization” score.

At event 320, the candidate probes are measured for probe performance for each target set, whether it be a validation composition target set or a reference composition target set. The results of the hybridization experiment are analyzed for a plurality of probe performance indicators which may include but are not limited to, slope of response curve (a differential response score), cross-hybridization, Y-axis intercept of response curve (equivalent to dye bias), reproducibility or noise, P-value of separability of distributions based on repeated measurements at two or more target copy number values, variance of signals, and variance of ratios.

At event 340, candidate probes are scored and/ranked according to the various indicators or parameters for probe performance. The candidate probes are scored for each target set tested.

At event 350, the experimental results obtained from each target set for each candidate probe, are compared to validate candidate probes across target sets.

At event 360, the candidate probes are evaluated for adequate differential response across target sets. For example, probes may be chosen that give the slope closest to the theoretical slope for the set of samples. This is accomplished by simple filtering, such as by selecting a range of tolerable ratios, or by using a more complex algorithm that uses the ratio information in conjunction with other probe information.

At event 370, candidate probes are evaluated for signal reproducibility across target sets. Signal reproducibility is determined in the same manner as with the self-self validation experiments described in event 250.

At event 80, candidate probes which have been validated by the probe metrics determined experimentally are passed to the next step of the selection process or may be selected for placement on a CGH array. Candidate probes which are not validated are discarded at event 90.

Referring now to FIG. 3, there is shown a flow chart of events that may be carried out in a nucleic acid probe selection method in accordance with the invention. At event 10, a nucleotide sample is selected for probe design for microarray analysis. The nucleotide sample may be a genome or genomic nucleotide range or ranges, such as a chromosome. At event 20, potential target sequences of the nucleotide sample of interest are identified, filtered and reduced to a set of appropriate target sequences for CGH and/or location analysis. The potential target sequences are filtered by size, number of repeat-masked bases and/or GC-content. Target sequences are also filtered and reduced in number by eliminating repetitive target sequences in event 20. Another parameter which can be used to filter target sequences, is to eliminate potential target sequences which comprise a restriction enzyme cut site. By limiting the size of the set of target sequences, the computational time needed to generate and analyze the candidate probes is decreased.

After determining a set of appropriate target sequences in event 20, candidate probes to the genomic sequence (e.g. chromosome) of interest are generated at event 30 as shown in FIG. 3. Generating a set of candidate probes comprises tiling probes across regions of the target sequences determined in event 20, which enables the candidate probes to be free of repeat-masked section as well as restriction cut sites if desired. The generation of candidate probes may.comprise additional filtering and reduction depending on the genomic sequence of interest.

At event 40, the candidate probes are filtered or reduced in total numbers by utilizing indicators or metrics of certain probe properties which assess candidate probe quality in silico. In silico means those parameters that can be determined without the need to perform any experiments, by using information is either calculated de novo or available from public or private databases. Probe parameters utilized to annotate candidate probes may include but are not limited to target specificity, thermodynamic properties, expression and association with genes, homology and also kinetic properties. Candidate probes which do not meet the in silico parameters or indicators for a “good” probe are discarded from the probe selection process at event 42.

Candidate probes which are identified to have certain desirable probe properties in silico, are subjected to a pairwise selection process to filter and reduce the number of potential probes at event 50. The pairwise filtering evaluates a pair of candidate probes for a probe property or set of property and scores the probes within the pair against each other according to the probe property analyzed. Probes which do not pass the pairwise selection process are not selected and are discarded in event 52. Probes which pass pairwise filtering may require further filtering and can be evaluated experimentally for other desirable probe properties at event 60. In certain embodiments, selecting probes for a CGH based array requires no further filtering or reduction of candidate probes besides those applied by the pairwise and in silico analysis as shown in event 54. As more indicators and metrics for probe performance are identified and adapted for analyzing probe performance in silico, less emphasis is placed on experimental probe results for CGH probe selection.

In the method shown in FIG. 3, candidate probes which meet the pairwise filtering may require further analysis by measuring specific “good” probe indicators/probe properties experimentally at event 60. To obtain a sense of a probe's performance, experiments are completed which measure properties of a probe that can, in the absence of more direct experiments, provide a good indication if a probe will be suitable for a CGH or location analysis array. Such experimentally measurable properties useful in determining a candidate probes performance include but are not limited to; raw signal intensity, reproducibility of signal intensity, dye bias, and susceptibility of non-specific binding.

Candidate probes which do not meet the experimentally measurable probe parameters are discarded/unselected in event 70, while the remaining candidate probes which meet the probe parameter standards in event 60 may be utilized for CGH arrays, event 72 or be subjected to further filtering by completing probe validation experiments at event 80. The order in which experimentally measurable probe parameters are applied to candidate probes may vary depending on the genomic sequence of interest.

At event 80, candidate probes are placed on an array and subjected to target sets/samples comprising known target sequences with known copy numbers, e.g., validation and reference compositions. The probes are evaluated and scored by assessing a plurality of probe properties over numerous target sets. The details of the probe properties and the methods utilized in probe validation experiments are described in more detail in FIG. 2 above.

The candidate probes are evaluated in event 80 for adequate signal response as well as reproducibility across target sets. The candidate probes which obtain a high validation score from the validation experiments are suitable for use on a CGH array, event 72, while candidate probes with deficient or poor validation scores are not selected in event 90.

Depending on the space available on the array chip, more or fewer probe parameters can be implemented and/or the thresholds and cut-offs of probe parameters may be adjusted as needed. Candidate probes may be prioritized, for example gene-by-gene, region-by region, or strictly filtered on validation scores. Also annotation of probes for position, gene association and expression may also be utilized to finalize the probe selection for a CGH or location array.

The overall system of probe design described above in which the subject methods of probe validation may be employed is further described in pending U.S. application Ser. No. ______ (Attorney Docket No. 10040115) the disclosure of which is herein incorporated by reference.

Methods of Producing an Array

The methods described above provide surface-bound polynucleotides with desirable binding characteristics. Once such surface-bound polynucleotides with desirable binding characteristics, i.e., “validated” surface-bound polynucleotides, have been identified, they may be used to fabricate an array. Accordingly, the invention provides a method of producing an array. In general, the method involves identifying a surface-bound polynucleotide with desirable binding characteristic, and fabricating an array containing that polynucleotide. A subject array may contain 1, 2, 3, more than about 5, more than about 10, more than about 20, more than about 50, more than about 100, more than about 200, more than about 500, more than about 1000, more than about 2000, more than about 5000 or more, usually up to about 10,000 or more, “validated” surface-bound polynucleotides.

Arrays can be fabricated using any means, including drop deposition from pulse jets or from fluid-filled tips, etc, or using photolithographic means. Either polynucleotide precursor units (such as nucleotide monomers), in the case of in situ fabrication, or previously synthesized polynucleotides (e.g., oligonucleotides, amplified cDNAs or isolated BAC, bacteriophage and plasmid clones, and the like) can be deposited. Such methods are described in detail in, for example U.S. Pat. No. 6,242,266, 6,232,072, 6,180,351, 6,171,797, 6,323,043, etc.

### Computer-Related Embodiments

The invention also provides a variety of computer-related embodiments. Specifically, the data analysis methods described in the previous section may be performed using a computer. Accordingly, the invention provides a computer-based system for analyzing data produced using the above methods in order to screen and identify surface-bound polynucleotides with desirable binding characteristics.

In certain embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor hereiri may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Kits

Also provided by the subject invention are kits for practicing the subject methods, as described above. The subject kits at least include a validation nucleic acid composition as described above. Other optional components of the kit include: nucleic acid labeling agents, such as for primer extension or nick translation and fluorescent labels conjugated to nucleotides. In some embodiments, arrays may be included in the kits. In alternative embodiments, the kit may also contain computer-readable media for performing the subject methods, as discussed above. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control analytes for use in testing the kit.

Utility

The subject methods find application in, among other applications, identifying surface-bound polynucleotides, e.g., BACs, cDNAs, oligonucleotides, etc., suitable for use in CGH assays, e.g., any application in which one wishes to compare the copy number of nucleic acid sequences found in two or more genomic samples. Once identified, surface-bound polynucleotides suitable for use in CGH assays may be used to make a CGH array. Such a CGH array may be used in CGH assays to obtain high quality, reliable, data that is free from the artifacts (e.g. compression of observed ratios due to crosshybridization of surface-bound polynucleotides with non-target sequences) commonly obtained using CGH arrays containing surface-bound polynucleotides identified using other methods. Accordingly, the subject methods find use in making CGH arrays. One type of representative application in which the subject CGH arrays find use is the quantitative comparison of copy number of one nucleic acid sequence in a first collection of nucleic acid molecules relative to the copy number of the same sequence in a second collection.

As such, the present invention may be used in methods of comparing abnormal nucleic acid copy number and mapping of chromosomal abnormalities associated with disease. In many embodiments, the subject methods are employed in applications that use polynucleotides immobilized on a solid support, to which differentially labeled nucleic acids produced as described above are hybridized. Analysis of processed results of the described hybridization experiments provides information about the relative copy number of nucleic acid domains, e.g. genes, in genomes.

Such applications compare the copy numbers of sequences capable of binding to the target elements. Variations in copy number detectable by the methods of the invention may arise in different ways. For example, copy number may be altered as a result of amplification or deletion of a chromosomal region, e.g. as commonly occurs in cancer.

Representative applications in which the subject methods find use are further described in U.S. PAT. Nos. 6,335,167; 6,197,501; 5,830,645; and 5,665,549; the disclosures of which are herein incorporated by reference.

The above discussion demonstrates a new method for screening for surface bound polynucleotides with desirable binding characteristics. Such methods are superior to currently used methods because they provide a way of testing CGH probes using chromosome mixtures of known composition. As such, the subject methods represent a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of assessing a candidate aCGH probe nucleic acid, said method comprising:
(a) contacting an array of surface-bound polynucleotides comprising said candidate aCGH probe nucleic acid with a validation nucleic acid composition, wherein said validation nucleic acid composition comprises either:
(i) a portion of an initial clone library that spans at least a portion of a genome; or
(ii) a portion of a genome in which all constituent members have substantially the same value for at least one physical parameter; and
(b) evaluating binding of said candidate aCGH probe nucleic acid to said validation composition of nucleic acids to assess said candidate aCGH probe nucleic acid.

2. The method according to claim 1, wherein said validation nucleic acid composition comprises a portion of an initial clone library that spans at least a portion genome.

3. The method according to claim 2, wherein said initial clone library is a contig library.

4. The method according to claim 2, wherein said initial clone library comprises clones that represent uniformly spaced regions of said genome.

5. The method according to claim 4, wherein said uniformly spaced regions of said genome are overlapping.

6. The method according to claim 4, wherein said uniformly spaced regions are not overlapping.

7. The method according to claim 2, wherein said initial clone library spans all of said genome.

8. The method according to claim 2, wherein said initial clone library comprises clones present in a chromosomal vector.

9. The method according to claim 2, wherein said validation nucleic acid composition is produced by:
separating an initial clone library into two or more subpopulations; and
selecting a subpopulation predicted to include a target that corresponds to said candidate aCGH probe nucleic acid as said validation nucleic acid composition.

10. The method according to claim 1, wherein said validation nucleic acid composition comprises a portion of a genome in which all constituent members have substantially the same value for at least one physical parameter.

11. The method according to claim 10, wherein said physical parameter is a parameter selected from the group consisting of: length, mass and charge-to-mass ratio.

12. The method according to claim 11, wherein said physical parameter is length.

13. The method according to claim 12, wherein a difference in lengths between any two members of said composition does not exceed about 100 nt.

14. The method according to claim 12, wherein said validation nucleic acid composition is produced by:
producing a population of fragment nucleic acids from an initial genomic sample;
separating said population of fragment nucleic acids into two or more subpopulations, wherein each of said subpopulations is made up of constituent members of substantially the same length; and
selecting a subpopulation predicted to include a target that corresponds to said candidate aCGH probe nucleic acid as said validation nucleic acid composition.

15. The method according to claim 1, wherein said evaluating comprises comparing a signal obtained from said candidate aCGH probe nucleic acid to a reference value.

16. The method of claim 1, wherein said candidate aCGH probe nucleic acid is an oligonucleotide.

17. The method according to claim 1, wherein said method is a method of assaying said candidate aCGH probe nucleic acid for suitability for use in array-based comparative genome hybridization assay.

18. The method of claim 17, wherein the method further comprises identifying a surface-bound polynucleotide suitable for use in array-based comparative genome hybridization assays.

19. The method of claim 1, wherein said array comprises a plurality of different candidate aCGH probe nucleic acids.

20. The method according to claim 1, further comprising determining a sequence of at least one candidate aCGH probe in silico.

21. A method of producing an array, comprising,
identifying by a method according to claim 1 an aCGH probe nucleic acid suitable for use in array-based comparative genome hybridization assay; and
fabricating an array comprising said surface-bound polynucleotide.

22. An array of surface-bound polynucleotides, wherein at least one of said surface-bound polynucleotide has been identified using the method of claim 1.

23. A computer-readable medium comprising:
programming for analyzing data provided by the method of claim 1.
